(19) United States
(12) Patent Application Publication
(10) Pub. No.: US 2011/0268741 A1
(43) Pub. Date: Nov. 3, 2011

(54) Immunotherapy for Contact Dermatitis Using Co-Signal Regulation

(75) Inventor: Koji Tamada, Baltimore, MD (US)

(73) Assignee: The Johns Hopkins University, Baltimore, MD (US)

(21) Appl. No.: 13/143,033

(22) PCT Filed: Jan. 5, 2010
(86) PCT No.: PCT/US10/20121

§ 371 Date: Jun. 30, 2011

Related U.S. Application Data
(60) Provisional application No. 61/142,478, filed on Jan. 5, 2009.

(51) Int. Cl. A61K 39/395; A61P 17/00; C07K 16/28

(52) U.S. Cl. 424/143.1; 530/388.1

(57) ABSTRACT

Compounds, compositions and methods for immunotherapy in contact dermatitis. Compounds and compositions (e.g. monoclonal antibodies) that stimulate the LAIR-1 expression pathway in LAIR-1 expressing immune cells and are useful for preventing or treating contact dermatitis are provided.

This application claims priority to U.S. provisional application No. 61/142,478, filed Jan. 5, 2009, which is incorporated herein by reference in its entirety. All other references cited herein are hereby incorporated by reference.

The invention disclosed herein was developed in part using support from a grant from the United States Department of Health and Human Services. The U.S. Government has certain rights in the invention.

### FIELD OF THE INVENTION

The invention relates to compounds, compositions and methods for immunotherapy in contact dermatitis. In particular, it relates to compounds that stimulate the LAIR-1 expression pathway in LAIR-1 expressing immune cells, e.g. monoclonal antibodies.

### BACKGROUND

Leucocyte-associated immunoglobulin-like receptor-1 (LAIR-1, CD305) is a type I transmembrane glycoprotein belonging to the immunoglobulin (Ig) superfamily and is expressed on most types of haematopoietic cells, including T cells, B cells, natural killer (NK) cells, monocytes, dendritic cells (DCs) and granulocytes.(1) Upon phosphorylation of two immunoreceptor tyrosine-based inhibitory motifs (ITIMs) in the cytoplasmic domain, LAIR-1 recruits Src homology 2 domain-containing tyrosine phosphatases (SHP) and/or C-terminal Src kinase (Csk), and exerts inhibitory effects on various immune cells (1-13). Recent studies have revealed that transmembrane and extracellular matrix collagens interact with LAIR-1 as its functional high-affinity ligands (10, 14). These findings collectively imply that the LAIR-1 inhibitory signal serves to set physiological thresholds for immune activation in order to prevent uncontrolled detrimental responses and to maintain peripheral self-tolerance, particularly in collagen-rich tissues. However, because of a lack of experimental models that manipulate LAIR-1 functions in vivo, this intriguing hypothesis has yet to be fully addressed. Allergic contact dermatitis, the third most common reason for outpatient dermatology visits, has a significant impact on a patient's quality of life (15). It is primarily caused by a delayed-type skin hypersensitivity reaction, in which sensitization and subsequent re-exposure to allergen elicits localized inflammatory reactions (16). Its molecular and cellular mechanisms have been investigated through ample studies employing contact hypersensitivity (CHS), an experimental model of allergic contact dermatitis (17-19). In the sensitization phase, epidermal Langerhans' cells (LCs) or dermal DCs take up antigens, migrate to draining lymph nodes (LNs), and present antigen to T cells to prime them. In the elicitation phase, sensitized T cells migrate to the skin and produce inflammatory mediators in response to Ag challenge. A recent study further suggested a potential role of NK cells in mounting a long-lived, hapten-specific CHS that is independent of T and B lymphocytes (20). Compelling evidence indicates that these responses are regulated by various immune regulators, including costimulatory signals, adhesion molecules, T helper 1 (Th1)- or T helper 2 (Th2)-type cytokines, and chemoattractive mediators (19). As collagen is the most abundant extracellular matrix component in the skin (21), its interactions with receptors are also expected to play a pathogenic role in CHS. Among known collagen receptors, a1b1 integrin positively regulates the elicitation phase of CHS by facilitating adhesion and extravasation of inflammatory cells (22). Genetic ablation of a1 integrin or administration of anti-a1 integrin-neutralizing monoclonal antibody (mAb), results in decreased responses of CHS.22 By contrast, the pathogenic role of LAIR-1 (another receptor of collagens) in CHS has yet to be explored.

In the invention set forth herein, transgenic mice expressing the LAIR-1-Ig decoy protein are disclosed, representing an experimental model for using to attenuate endogenous LAIR-1 in vivo. By applying CHS in LAIR-1-Ig transgenic mice, the pathogenic functions of LAIR-1 in allergic contact dermatitis were explored, resulting in the development of compositions and methods for treating contact dermatitis.

### BRIEF DESCRIPTION OF THE DRAWINGS

FIG. 1. Generation of leucocyte-associated immunoglobulin-like receptor-1 (LAIR-1)-Ig transgenic mice. (a) A schematic map of the chimeric LAIR-1-Ig gene is shown. The complementary DNA (cDNA) of the mouse LAIR-1 (mLAIR-1) extracellular domain fused with the human IgG1 constant (Fc) region was cloned into an expression plasmid containing an actin promoter, an intron and a 30 untranslated region (UTR). The fragment excised at CalI sites was microinjected into fertilized eggs. (b) The concentration of serum LAIR-1-Ig protein was determined in LAIR-1-Ig transgenic mice (d) or in control littermates (s), at 4-6 weeks of age, using enzyme-linked immunosorbent assay (ELISA) specific to human IgG. Each symbol represents a value of an individual mouse. (c) In vivo distribution of transgenic LAIR-1-Ig protein was detected by immunohistochemistry using monoclonal anti-human IgG in the indicated organs. As a control, tissues from wild-type B6 mice were processed similarly. (d) Earlobes of wild-type or LAIR-1-Ig transgenic mice were stained with biotin-conjugated LAIR-1-Ig protein followed by detection with streptavidin-horseradish peroxidase (HRP), in order to examine the potential of endogenous LAIR-1 ligands to interact with LAIR-1.

FIG. 2. Exacerbated contact hypersensitivity (CHS) by blockade of the leucocyte-associated immunoglobulin-like receptor-1 (LAIR-1) signal. (a) LAIR-1-Ig transgenic mice () or control littermates (∘) were sensitized with dinitrofluorobenzene (DNFB) on days 0 and 1, and challenged at earlobes on day 5. Net ear swelling was measured at the indicated time-points after DNFB challenge. Each symbol represents an individual earlobe, and average values±standard deviations (SDs) are shown as horizontal and vertical bars respectively. Accumulated data from two independently repeated experiments are shown. (b) Earlobes were harvested from LAIR-1-Ig transgenic mice or from control littermates 1 day after DNFB challenge and stained with haematoxylin and eosin (H & E) (magnification·400).

FIG. 3. A role of leucocyte-associated immunoglobulin-like receptor-1 (LAIR-1) in the sensitization and elicitation phases of contact hypersensitivity (CHS). (a and b) LAIR-1-Ig transgenic mice () or control littermates (∘) were sensitized with dinitrofluorobenzene (DNFB) on days 0 and 1, and draining lymph nodes (LNs) were harvested on day 5. LN cells were also harvested from transgenic mice (▪) and from control littermates (□) that had not been sensitized with DNFB. These cells were incubated with DNBS in vitro, and proliferative activity on day 3 [expressed as counts per minute (CPM)] (a) and interferon-γ (IFN-γ) production in the culture with 30 μg/ml of DNBS (b) was examined. (c) B6 mice were sensitized with DNFB on days 0 and 1, and treated with 200 μg of LAIR-1-Ig (d) or control Ig (s) on days) 1, 0 and 2. On day 5, T cells purified from draining LN cells of these mice were transferred into naïve B6 recipients, which were subsequently challenged with DNFB; ear thickness measurements were taken 24 hr later. (d) B6 mice were sensitized with DNFB on days 0 and 1, and the draining LN cells were harvested on day 5. These LN cells were adoptively transferred into B6 mice, which had been treated with LAIR-1-Ig () or control Ig (∘) 1 day before. These recipient mice were then challenged with DNFB on the earlobes and the net swelling was measured 24 hr later. In (c) and (d), each symbol represents an individual earlobe, and the averages±standard deviations (SD) are shown as horizontal and vertical bars respectively. (e) B6 mice were sensitized with DNFB on days 0 and 1, and challenged on the earlobes on day 5. On day 6, lymphocytes infiltrating the earlobes of the mice were harvested and stained with monoclonal anti-LAIR-1 (solid line) or control IgG (dotted line) together with monoclonal anti-CD3. LAIR-1 expression on T cells gated as CD3-positive was analyzed by flow cytometry and the data are shown on a histogram. All experiments were repeated at least twice and representative results are shown.

FIG. 4. Inhibitory effects and mechanisms of leucocyte-associated immunoglobulin-like receptor-1 (LAIR-1) signalling in T-cell responses. (a) Spleen cells from naïve B6 mice were stained with monoclonal anti-CD44, anti-CD4 or CD8, and with monoclonal anti-LAIR-1 (filled histogram) or control Ig (open histogram). LAIR-1 expression on CD44^{low} naïve T cells was analyzed using flow cytometry (left panels). Spleen cells were stimulated with immobilized monoclonal anti-CD3 (3 μg/ml) and with soluble monoclonal anti-CD28 (1 μg/ml) in vitro for 3 days, and then stained with monoclonal anti-CD69, monoclonal anti-CD4 or CD8, and monoclonal anti-LAIR-1 (filled histogram) or control Ig (open histogram). LAIR-1 expression on CD69+-activated T cells was analyzed (right panels). (b-e) Spleen T cells isolated as a Thy-1.2+ population were stimulated by immobilized monoclonal anti-CD3 (at the indicated doses in b, and at 10 μg/ml in c-e) in the presence of co-immobilized monoclonal anti-LAIR-1 (d) or control IgG (s). Proliferative activity on day 3 (b) and the production of interferon-γ (IFN-γ), interleukin (IL)-2, IL-4 and IL-10 in the culture supernatants (c) was assessed. In (d), T cells were labelled with carboxyfluorescein succinimidyl ester (CFSE) before culture, and cellular division on day 4 was measured using a dilution of CFSE. The percentages of cells showing more than one division are shown. In (e), cell cycle progression was analyzed on day 2 using a 5-bromo-2-deoxyuridine (BrdU) flow kit. The percentages of cells in apoptosis (lower left square), and in G0/G1 (lower middle square), S (upper square) and G2/M (lower right square) phases are indicated. Representative data from at least three independent experiments are shown.

FIG. 5. Leucocyte-associated immunoglobulin-like receptor-1 (LAIR-1) expression and function in memory T cells. (a) Spleen cells from naïve B6 mice were stained with monoclonal anti-CD4 or CD8, monoclonal anti-CD44, monoclonal anti-CD62L and monoclonal anti-LAIR-1 (filled histogram) or control Ig (open histogram). Expression of LAIR-1 on central memory (CD44^{high}, CD62L^{high}) and effector memory (CD44^{high}, CD62L^{low}) cells in CD4⁺ or CD8⁺ T-cell subsets were analyzed using flow cytometry. (b) Purified CD44^{low} naïve T cells (1.6·10⁶ cells/ml) were stimulated with immobilized monoclonal anti-CD3 (40 μg/ml for CD4⁺ T cells, 20 μg/ml for CD8⁺ T cells) in the presence of 15 μg/ml of co-immobilized monoclonal anti-LAIR-1 (filled column) or control Ig (open column) for 96 hr. (c) Purified CD44^{high} memory T cells (1.4·10⁶ cells/ml in CD4⁺ T cells, 1.0·10⁶ cells/ml in CD8⁺ T cells) were stimulated with immobilized monoclonal anti-CD3 (2.5 μg/ml for CD4⁺ T cells, 1.25 μg/ml for CD8⁺ T cells) in the presence of 15 μg/ml of coimmobilized monoclonal anti-LAIR-1 (filled column) or control Ig (open column) for 48 hr. In both (b) and (c), the production of interferon-γ (IFN-γ) in the culture supernatants was measured using specific enzyme-linked immunosorbent assay (ELISA). Representative data from at least three independent experiments are shown.

FIG. 6. The expression and inhibitory functions of leucocyte-associated immunoglobulin-like receptor-1 (LAIR-1) on dendritic cells (DCs). (a) Freshly isolated Langerhans' cells (LCs), gated as a CD11c⁺ population in epidermal cells, were stained with monoclonal anti-LAIR-1 (filled histogram) or control Ig (open histogram), and analyzed using flow cytometry. (b) Immature spleen DCs isolated as CD11c⁺ were stained with monoclonal anti-LAIR-1 and monoclonal anti-CD86, and analyzed using flow cytometry (left panel). Immature spleen DCs were stimulated with lipopolysaccharide (LPS) in the presence of interleukin (IL)-4 and granulocyte-macrophage colony-stimulating factor (GM-CSF). After 2 days, the expression of LAIR-1 and of CD86 was analyzed (right panel). (c) Purified spleen DCs were stimulated in vitro with the indicated doses of cytosine-phosphate-guanosine (CpG) plus interferon-γ (IFN-γ), IL-4 and GM-CSF in the presence of immobilized monoclonal anti-LAIR-1 (filled bar) or control Ig (open bar). After 24 hr, the level of IL-12 p70 in the supernatants was measured. (d) DCs generated from bone marrow (BM) cells were cultured with an immobilized collagen III or control bovine serum albumin (BSA) in the presence or absence of LPS. A soluble form of monoclonal anti-LAIR-1 (as a blocking agent), or control Ig, was also included in the culture. After 24 hr, IL-6 production in the supernatants was measured. (e) Fluorescein isothiocyanate (FITC) was painted on the shaved abdomen of LAIR-1-Ig transgenic mice or control mice. After 24 hr, draining LNs were harvested and stained with CD11c and major histocompatibility complex (MHC) class II. The percentages of FITC-positive cells in CD11c/MHC class II-double positive DCs were assessed using flow cytometry. Representative data from at least three independent experiments are shown. *P<0_{—}05. ND, not detected; Tg, transgenic.

FIG. 7. Leucocyte-associated immunoglobulin-like receptor-1 (LAIR-1) functions on natural killer (NK) cells in vitro and in vivo. (a) Spleen cells from B6 RAG-deficient mice were stained with monoclonal anti-NK1.1, together with monoclonal anti-LAIR-1 (filled histogram) or control Ig (open histogram). LAIR-1 expression on NK1.1+ cells was analyzed using flow cytometry. (b) NK cells isolated from B6 RAG-deficient mice were stimulated with immobilized monoclonal anti-NK1.1 plus co-immobilized monoclonal anti-LAIR-1 (▪) or control IgG (□) in the presence of interleukin-2 (IL-2). As a negative control, they were also cultured without IL-2 (∘). Production of interferon-γ (IFN-γ) was assessed at the indicated timepoints. (c) B6 RAG-deficient mice were treated with LAIR-1-Ig fusion protein () or with control human IgG (∘) on days) 1 and 3. On days 0 and 1, these mice were sensitized with dinitrofluorobenzene (DNFB) and challenged at the earlobes on day 5. Net ear swelling was measured 1, 2 and 3 days after DNFB challenge. Each symbol represents an individual earlobe, and the average values±standard deviations (SDs) are indicated as horizontal and vertical bars respectively. Data accumulated from two independently repeated experiments are shown.

### DESCRIPTION OF THE INVENTION

The inventor has discovered a novel immune regulatory pathway that plays a crucial role for the inhibition of anti-allergen immune responses, thereby enabling the treatment of allergen caused contact dermatitis using specific monoclonal antibodies that target this pathway.

Accordingly, it is one object to provide a monoclonal antibody that specifically interacts with a component of and stimulates a LAIR-1 expression pathway in a LAIR-1 expressing immune cell. LAIR-1 expressing immune cells include, for example, T lymphocytes, B lymphocytes, Natural Killer (NK) cells, Dendritic cells (DC), Macrophages, and granulocytes.

The monoclonal antibody may be an anti-LAIR-1 antibody. It may also be an antibody directed against other components in the LAIR-1 expression pathway, for example recombinant protein of LAIR-1 ligand, small molecules and DNA/RNA aptamers that bind LAIR-1, ribozymes, antisense DNA, siRNA, and micro RNA specific for nucleic acids encoding LAIR-1. Means of making such monoclonal antibodies are familiar to those of skill in the art.

In general, the LAIR-1 expression pathway and antibodies directed thereto will be in a mammal, including, but not limited to a rodent (for example a mouse) cat, dog, horse, or human or nonhuman primate. Methods and procedures described hereinbelow are suitable for making the appropriate monoclonal antibodies and pharmaceutical compositions and administering them according to the claimed methods.

Also provided is a pharmaceutical composition comprising the monoclonal antibodies as detailed above. Thus, the invention includes a pharmaceutical composition comprising monoclonal anti-LAIR-1, in particular directed at mammalian LAIR-1, especially human LAIR-1.

The monoclonal antibodies and pharmaceutical compositions will be useful for treatment of contact dermatitis caused by allergen(s) in subjects afflicted with such contact dermatitis. Thus, a method is provided for treating a contact dermatitis caused by an allergen comprising administering a monoclonal antibody as described above or pharmaceutical composition containing same to a subject in need of treatment.

By “subject” is meant any animal that is capable of suffering a contact dermatitis caused by an allergen, in particular a mammal, e.g. a human. A subject may also be referred to as a patient.

Allergens which cause contact dermatitis for which the treatment may be effective include, but are not limited to, substances from plants such as poison ivy, certain metals such as nickel, rubber products, and chemicals.

Dosages of the aforementioned monoclonal antibodies and compositions thereof can be determined by those of skill in the art without undue experimentation. Dosages are expected to be in the range of 0.001 to 10 mg/kg of body weight, typically 0.1-5 mg/kg, depending on route of administration, and the concentration/amount that is delivered to a target site (e.g. topically to skin).

Routes of administration include suitable methods known to those of skill in the art, for example topical; oral; intravenous, intramuscular, subcutaneous, nasal, rectal and other suitable means known to those of skill in the art. Typically, topical compounds deliverable through skin will be used.

Compositions and formulations for administration are known to those of skill in the art. Formulations may include pharmaceutically acceptable diluents, excipients and carriers known to persons of skill in the art as being compatible with the mAbs, and suitable for local or systemic administration to an animal, particularly a human or other mammal, according to the invention. Typical excipients, diluents or carriers include physiological saline or phosphate buffered saline for intravenous, intramuscular, subcutaneous injections and ointment excipients such as mineral oil, paraffin, propylene carbonate, white petrolatum and white wax for topical administration. Useful solutions for oral or parenteral administration can be prepared by any of the methods well known in the pharmaceutical arts, described, for example, in Remington's Pharmaceutical Sciences, (Gennaro, A., ed.), Mack Pub., (1990), incorporated herein by reference, in particularly for the description of such diluents, excipients and carriers.

Thus, the invention includes mAbs formulated into compositions which optionally include suitable diluents, excipients and carriers as known in the art for administration in the claimed methods.

By “pharmaceutically acceptable diluents, excipients and carriers” is meant such compounds as will be known to persons of skill in the art as being compatible with the pharmaceutical compositions and suitable for local or systemic administration to an animal, particularly a human or other mammal, according to the invention.

As used herein, the terms “treatment,” “treating,” etc., refer to obtaining a measurable pharmacologic and/or physiologic effect, e.g. a diminution of the symptoms of a contact dermatitis caused by an allergen.

As used herein, the terms “prevention” and “prophylaxis” refer to administering the compounds/compositions of the invention in advance of exposure or in advance the development of symptoms requiring relief. The compounds, compositions and methods of the invention can be used for the prophylaxis or prevention of contact dermatitis if administered prior to exposure or to the development of symptoms.

The term “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and is compatible with other ingredients of the formulation. For example, the carrier for a formulation containing the present therapeutic compounds and compositions preferably does not include oxidizing agents and other compounds that are known to be deleterious to such. Suitable carriers include, but are not limited to, water, dextrose, glycerol, saline, ethanol, buffer, dimethyl sulfoxide, Cremaphor EL, and combinations thereof. The carrier may contain additional agents such as wetting or emulsifying agents, or pH buffering agents. Other materials such as anti-oxidants, humectants, viscosity stabilizers, and similar agents may be added as necessary.

Pharmaceutically acceptable salts herein include the acid addition salts (e.g. formed with a free amino group) and which are formed with inorganic acids, including, but not limited to hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, and histidine.

The term “pharmaceutically acceptable excipient,” includes vehicles, adjuvants, or diluents or other auxiliary substances, such as those conventional in the art, which are readily available to the public. For example, pharmaceutically acceptable auxiliary substances include pH adjusting and buffering agents, tonicity adjusting agents, stabilizers, wetting agents and the like.

As used herein, the singular forms “a”, “an”, and “the” include plural forms unless the context clearly dictates otherwise. Thus, for example, reference to “a compound” includes a plurality of such compounds.

As mentioned above, effective amounts of the pharmaceutical compounds are administered to an individual or subject, where “effective amount” means a dosage sufficient to produce a desired result. In some embodiments, the desired result is a diminution or complete alleviation of the symptoms of contact dermatitis. In other embodiments, the desired result is the prevention of symptoms (prophylaxis).

Typically, the compositions to be used in the instant invention will contain from less than about 1% up to about 99% of the active ingredient(s), e.g. the monoclonal antibodies. The appropriate dose to be administered depends on the subject to be treated, such as the general health of the subject, the age of the subject, the state of the disease or condition, the weight of the subject, etc. A typical dose of monoclonal antibody, is expected to be 0.1-5 mg/kg of body weight.

The pharmaceutically acceptable excipients, such as vehicles, carriers or diluents, are conventional in the art. Suitable excipient vehicles are, for example, water, saline, dextrose, glycerol, ethanol, or the like, and combinations thereof. In addition, if desired, the vehicle may contain minor amounts of auxiliary substances such as pH adjusting and buffering agents, tonicity adjusting agents, stabilizers, wetting agents or emulsifying agents. Actual methods of preparing such dosage forms are known, or will be apparent, to those skilled in the art. See, e.g., Remington: The Science and Practice of Pharmacy (University of the Sciences in Philadelphia, 2005). The composition or formulation to be administered will, in any event, contain a quantity of the agent adequate to achieve the desired state in the individual being treated.

The therapeutic compounds can be formulated into preparations for administration by dissolving, suspending or emulsifying them in an aqueous or non-aqueous solvent, such as vegetable or other similar oils, including corn oil, castor oil, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Patents and other publications cited herein are hereby incorporated by reference.

The following abbreviations are used throughout the description:

APC, antigen-presenting cell; B6, C57BL/6; BM, bone marrow; CHS, contact hypersensitivity; Csk, C-terminal Src kinase; DC, dendritic cell; DNBS, dinitrobenzene sulfonic acid; DNFB, dinitrofluorobenzene; FcR, Fc receptor; FITC, fluorescein
isothiocyanate; GM-CSF, granulocyte-macrophage colony-stimulating factor; HRP, horseradish peroxidase; IFN-γ, interferon-γ; IL, interleukin; ITIM, immunoreceptor tyrosine-based inhibitory motif; LAIR-1, leucocyte-associated immunoglobulin-like receptor-1; LC, Langerhans' cell; LN, lymph nodes; LPS, lipopolysaccharide; mAb, monoclonal antibody; NK, natural killer; SHP, Src homology 2 domain-containing tyrosine phosphatases; Th1, T helper 1; Th2, T helper 2; Treg, T regulatory; UTR, untranslated region.

### Materials and Methods

### LAIR-1-Ig Transgenic Mice

The extracellular domain of mouse LAIR-1 was cloned from complementary DNA (cDNA) of mouse spleen cells and fused in-frame with the Fc region of human immunoglobulin G1 (IgG1) (LAIR-1-Ig), as previously reported (23). The LAIR-1-Ig gene was then cloned into the expression plasmid containing the actin promoter, an intron and the 30 untranslated region (UTR). The entire construct was excised from the plasmid and microinjected into the fertilized eggs of FVB mice, as previously described (24). The transgenic founders were identified by detecting the LAIR-1-Ig gene by Southern blot analysis as well as by measuring LAIR-1-Ig protein in serum using human IgG-specific enzyme-linked immunosorbent assays (ELISAs). The transgenic mice were backcrossed with C57BL/6 (B6) mice for at least six generations. The in vivo distribution of LAIR-1-Ig and its decoy effect were assessed using immunohistochemical staining (25). Briefly, frozen sections from various organs were fixed in acetone, followed by a blockade of non-specific protein binding and endogenous peroxidase activity. Then, sections were incubated with 5 μg/ml of biotin-conjugated anti-human IgG (Rockland, Gilbertsville, Pa.), washed and stained with streptavidin-conjugated horseradish peroxidase (HRP) (DakoCytomation, Carpinteria, Calif.). In some experiments, tissue samples were stained with 0.5 μg/ml of biotin-conjugated LAIR-1-Ig followed by streptavidin-HRP. Staining was developed with diaminobenzidine using a commercial staining kit (LASB+kit; DakoCytomation) according to the manufacturer's instructions.

### Mice and Reagents

B6 mice and B6 RAG2-deficient mice were purchased from the National Cancer Institute and Taconic Inc. (Hudson, N.Y.), respectively. In all experiments, age- and gender-matched 6-10-week-old mice were used. The mice were maintained in the animal facility under protocols approved by the Institutional Animal Care and Use Committee. Mouse LAIR-1-human Ig fusion protein, which forms a homodimer through an intermolecular disulfide bond and thus exists as a bivalent structure (data not shown), was purified from the culture supernatants of Chinese hamster ovary cells stably transfected with the LAIR-1-Ig expression vector. The capability of the LAIR-1-Ig protein to compete against interactions between collagen and endogenous LAIR-1 was confirmed, as it attenuated binding of fluorescein isothiocyanate (FITC)-conjugated collagen with LAIR-1-expressing cells (data not shown). Two anti-LAIR-1 mAb-producing hybridomas, clones DK3.4 and DKR431, were independently generated from LAIR-1-Ig-immunized Armenian hamster and Lewis rat, respectively, using a standard method.26 DK3.4 was used as an immobilized form to deliver LAIR-1 signals in vitro. By contrast, DKR431 was used for staining in a flow cytometric analysis. Control hamster IgG, rat IgG and human IgG were purchased from Rockland or Sigma-Aldrich (St Louis, Mo.).

### Assay for CHS

CHS induced by dinitrofluorobenzene (DNFB; Sigma-Aldrich) was conducted as previously reported.27 Briefly, the mice were sensitized by painting 25 μl of 0.5% DNFB dissolved in acetone/olive oil mixture (4:1, v/v) on the shaved abdomen. One day later, the same sensitization procedure was repeated. Five days after the first sensitization, the mice were challenged with 10 μl of 0.2% DNFB on each side of their earlobes. The ear thickness was measured under anaesthesia using a thickness gauge (model 21-790-1; Käfer Messuhrenfabrik, Villingen-Schwenningen, Germany) at 0.01 mm resolution. Measurements were performed 1, 2, 3 and 4 days after challenge using an investigator blinded to the experimental groups, and the net increase was calculated by subtracting ear thickness before challenge from that after challenge in individual earlobes. In representative mice, ear tissues were harvested 24 hr after DNFB challenge, fixed in formalin and embedded with paraffin. The sections were stained with haematoxylin and eosin (H & E) for pathological analysis. To analyze T-cell priming in CHS in LAIR-1-Ig transgenic mice, draining axillary and inguinal LNs were harvested 5 days after the first sensitization. These LN cells (1.5·10⁶ cells/ml) were incubated in the presence of dinitrobenzene sulphonic acid (DNBS; Sigma-Aldrich), and the proliferation and interferon-γ (IFN-γ) production was assessed by the incorporation of [³H]thymidine and using ELISA kits (eBioscience, San Diego, Calif.), respectively. For the transfer of primed T cells, B6 mice were sensitized with DNFB on days 0 and 1, and were also injected intraperitoneally (i.p.) with 200 μg of LAIR-1-Ig protein on days 1, 0 and 2. On day 5, T cells were purified from the draining LNs of these mice and transferred into the nave B6 mice at 5·10⁷ cells per mouse. One hour after transfer, the recipient mice were challenged with DNFB and ear thickness was measured 24 hr later. For analysis of the elicitation phase of CHS, draining LN cells (5·10⁷ cells) of the DNFB-sensitized B6 mice were transferred intravenously (i.v.) into recipient mice that had been pretreated i.p. with 200 μg of LAIR-1-Ig fusion protein, or control human IgG, 1 day previously. One hour after transfer of LN cells, the mice were challenged with DNFB, and the net increase of ear thickness was assessed 24 hr later. In vitro analysis of T-cell functions T cells were isolated from B6 spleen cells by using anti-Thy-1.2 microbeads (Miltenyi Biotec, Bergisch Gladbach, Germany). Isolated cells (1.5·10⁶ cells/ml) were stimulated with the indicated doses of immobilized anti-CD3 mAb (BD Biosciences, San Jose, Calif.) in the presence of 15 μg/ml of co-immobilized anti-LAIR-1 mAb (DK3.4) or control hamster IgG. Proliferative activity was assessed by measurement of [³H]thymidine uptake during the last 15 hr of the 3-day culture period. The production of IFN-γ, interleukin (IL)-2, IL-4 and IL-10 in the culture supernatants was measured using specific ELISA kits (eBioscience). To monitor cell division, T cells were labelled with carboxyfluorescein succinimidyl ester (CFSE) (Molecular Probes, Eugene, Oreg.) before cell culture and a dilution of CFSE intensity along with the cell division was evaluated using flow cytometry. Cell cycle progression and the level of apoptosis were assessed using the 5-bromo-2-deoxyuridine (BrdU) flow Kit (BD Biosciences), according to the manufacturer's instructions.

Isolation of Naïve and Memory T Cells

Naïve and memory CD4⁺ and CD8⁺ T cells were isolated from mouse spleen and lymph node cells using a CD4⁺ or a CD8⁺ T-cell isolation kit (Miltenyi Biotec) and then sorted into CD44^{low} and CD44^{high} cells using a FACS Vantage (BD Biosciences). Purity of the samples was routinely tested after sorting and was more than 96%. Central and effector memory T cells were identified as CD62L^{high} and CD62L^{low} cells within CD44^{high} T cells, respectively. Isolated naïve and memory T cells were stimulated with the indicated doses of immobilized anti-CD3 mAb (BD Biosciences) in the presence of 15 μg/ml of co-immobilized anti-LAIR-1 mAb (DK3.4) or control IgG After 2-4 days, production of IFN-γ in the culture supernatants was measured using ELISA kits (eBioscience).

### Analysis of LCs and T Cells in Earlobes

Epidermal cell suspensions containing LCs were prepared as described previously (28). Briefly, skin sheets from earlobes were removed and floated in 1% (for the ventral halves) or 0.33% (for the dorsal halves) trypsin in Hanks' balanced salt solution (HBSS) for 30-60 min at 37° C. Epidermis was then separated from dermis, using forceps, in RPMI medium supplemented with 10% fetal calf serum. Epidermal cell suspensions were prepared by vigorous pipetting and filtration through nylon mesh. The expression of proteins on LCs was analyzed using a flow cytometer under the gate of CD11c⁺ populations in epidermal cell suspensions. A suspension of earlobe-derived cells was generated by mincing the earlobes into small pieces followed by incubation at 37° C. in a medium containing 400 U/ml of liberase blendzyme 2 (Roche Applied Science, Indianapolis, Ind.) for 60 min. LAIR-1 expression on CHS skin-infiltrating T cells was analyzed under a gate of CD3⁺ cells by flow cytometry.

### Analysis of DC Functions

Isolation of spleen DC and generation of bone marrow (BM)-derived DC were conducted as previously reported.29 Briefly, for spleen DC, B6 spleens were dissected into small pieces and incubated for 60 min at 37° C. in a medium containing 400 U/ml of liberase blendzyme 2. The cell suspension was obtained by vigorous pipetting and passed through a nylon filter. Spleen DCs were purified by anti-CD11c microbeads according to the manufacturer's instructions (Miltenyi Biotec). In some experiments, the isolated immature DCs (1·10⁵ cells/ml) were cultured in the presence of 10 ng/ml of IL-4, 20 ng/ml of granulocyte-macrophage colony-stimulating factor (GM-CSF) (R&D Systems, Minneapolis, Minn.) and 1 μg/ml of lipopolysaccharide (LPS) (Sigma-Aldrich) for 2 days, in order to generate mature DCs. To trigger IL-12 production, immature spleen DCs (1·10⁵ cells/ml) were incubated with IFN-γ (20 ng/ml), IL-4 (10 ng/ml), GM-CSF (20 ng/ml) and the graded doses of cytosine-phosphate-guanosine (CpG), as previously reported, 30 in the presence of 20 μg/ml of immobilized anti-LAIR-1 mAb (DK3.4) or control IgG. The concentration of IL-12 p70 in the culture supernatants was measured using an ELISA kit (eBioscience). To generate BM-derived DCs, B6 BM cells were cultured for 7 days in a medium supplemented with 3 ng/ml of GM-CSF. IL-6 production was induced by incubating BMderived DCs (1·10⁵ cells/ml) with 1 ng/ml of LPS in the presence of 10 μg/ml of immobilized collagen III or control bovine serum albumin (BSA). Soluble anti-LAIR-1-blocking mAb (DK3.4) or control hamster IgG was also included in the culture at 40 μg/ml. The concentration of IL-6 in the culture supernatants was measured using ELISA kits (eBioscience). For the migration assay of skin DCs, 400 μl of 0.5% FITC (Sigma-Aldrich) dissolved in an acetone/dibutylphtalate mixture (1:1, v/v) was painted onto the shaved abdomen of LAIR-1-Ig transgenic mice or control mice. One day later, axillary and inguinal LNs were harvested, and the level of FITC expression on CD11c/major histocompatibility complex (MHC) class II doublepositive DCs was assessed using flow cytometry.

### Analysis of NK-Cell Functions

NK cells were isolated from B6 RAG-2-deficient mice by collecting a non-adherent population of spleen cells, as previously described (31). Isolated NK cells (5·10⁵ cells/ml) were stimulated with 10 μg/ml of immobilized anti-NK1.1 mAb (clone PK136) plus 15 μg/ml of co-immobilized anti-LAIR-1 mAb (DK3.4) or control hamster IgG, in the presence or absence of 150 IU/ml of recombinant human IL-2 (Chiron, Emeryville, Calif.). The production of IFN-γ was assessed using a specific ELISA kit (eBioscience). To assess LAIR-1 functions in NK cell-induced CHS, B6 RAG-2-deficient mice were sensitized and challenged with DNFB, as described above. One day before and 3 days after the first sensitization, the RAG-2-deficient mice were injected i.p. with 250 μg of LAIR-1-Ig fusion protein or control IgG. Ear thickness was measured 24 hr after DNFB challenge.

### Statistical Analysis

Statistical significance, measured using a two-sided paired Student's t-test or the non-parametric sign test when the normality was not plausible, was calculated using Excel v2003 (Microsoft, Redmond, Wash.) or S-plus, based on the number of experiments indicated in the figure legends. Differences were considered to be significant at P<0.05.

### Example 1

### Generation of LAIR-1-Ig Transgenic Mice

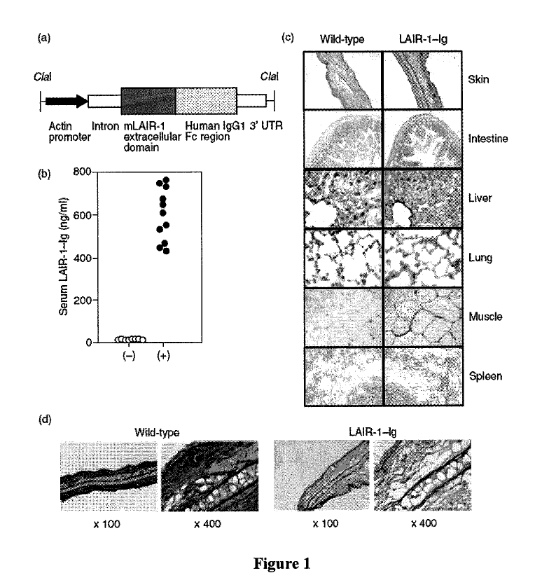

Initially, a chimeric gene of the extracellular domain of mouse LAIR-1 fused with the human IgG Fc region (LAIR-1-Ig) was constructed, which was then cloned into an expression vector containing the actin promoter, intron and the 30 UTR (FIG. 1a). In the transgenic mice of this construct, LAIR-1-Ig protein was ubiquitously expressed by the actin promoter and was readily detected as a soluble protein in the serum (FIG. 1b). Histochemical analyses detected LAIR-1-Ig in the collagen-rich connective tissues in various organs (e.g. dermis, basement membrane and muscle fascicle) (FIG. 1c). The association between LAIR-1-Ig and host Fc receptor (FcR) appeared to be negligible, because no LAIR-1-Ig was detected in the B-cell area of the transgenic spleen. This notion was further supported by our BIAcore surface plasmon resonance data that indicated a very weak interaction between human IgG and mouse FcR (data not shown). The staining of collagen-rich tissues by biotin-conjugated LAIR-1-Ig was weaker in the transgenic mice than in the wildtype mice (FIG. 1d), suggesting that transgene-derived LAIR-1-Ig protein occupies the binding site of LAIR-1 ligands in vivo. Taken together, LAIR-1-Ig protein is systemically and constitutively expressed in LAIR-1-Ig transgenic mice and serves as a decoy by competing with the interaction of LAIR-1 with its ligands.

LAIR-1-Ig transgenic mice are viable at birth, develop normally and are able to reproduce. Major organs (e.g. brain, lung, heart, liver, kidney, small and large intestines, muscles, skin, spleen and lymph nodes) are normal at least at the level of gross observations, and no abnormalities were found in the cellular compositions of immune organs when analyzed using flow cytometry. While two LAIR-1-Ig transgenic mice (lines 1841 and 1843), were generated by independent microinjections and backcrossing with B6 mice, line 1843 was mainly used in this study because both lines produce similar levels of serum LAIR-1-Ig and show the same phenotypes as far as could be established.

### Example 2

### An Inhibitory Role of LAIR-1 in CHS Pathogenesis

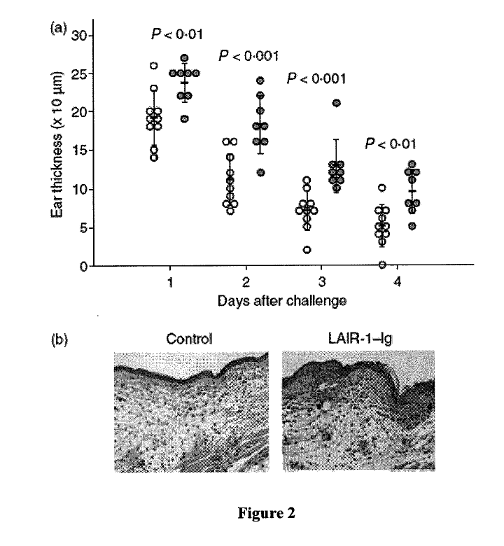

The susceptibility of LAIR-1-Ig transgenic mice to CHS, an experimental model of allergic contact dermatitis, was then assessed. Sensitization and subsequent elicitation with DNFB resulted in an exacerbated ear swelling in LAIR-1-Ig transgenic mice, compared with control littermates, which was statistically significantly different for at least 4 days (FIG. 2a). The mice without sensitization or elicitation developed no ear swelling (data not shown), suggesting that these responses were specific to DNFB. In pathological analyses, ear tissues of DNFB-treated LAIR-1-Ig transgenic mice showed accelerated inflammation, and thickening of the epidermis was accompanied by hyperkeratosis and epidermal oedema, together with a massive infiltration of inflammatory cells in the dermis (FIG. 2b). LAIR-1-Ig transgenic mice also demonstrated severe CHS in response to oxazolone, another type of hapten (data not shown). These findings suggest that interruption of the interaction of LAIR-1 with its ligands in the LAIR-1-Ig transgenic mice results in an exacerbated CHS.

### Example 3

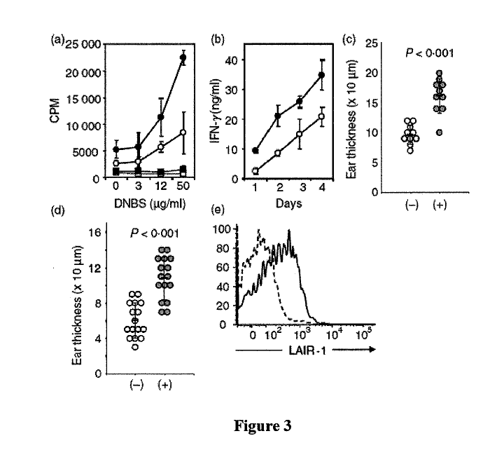

We next examined which phase of CHS—sensitization, elicitation, or both—is regulated by LAIR-1 functions. First, to assess the sensitization phase, draining LN cells from the DNFB-sensitized LAIR-1-Ig transgenic mice or from control mice were harvested before elicitation of CHS and were restimulated in vitro with DNBS, a watersoluble form of DNFB. Proliferation and IFN-γ production of the draining LN cells from LAIR-1-Ig transgenic mice were significantly higher than those from control littermates (FIG. 3a,b). These responses were specific to DNFB, because the mice that were not sensitized showed no detectable proliferation (FIG. 3a) or IFN-γ production (data not shown). These results suggest that interruption of LAIR-1 functions in the sensitization phase of CHS promotes the priming of hapten-specific T cells. To confirm this, T cells were isolated from the draining LNs of the mice sensitized with DNFB under LAIR-1-Ig or control Ig treatment and were adoptively transferred into naive recipient mice. Elicitation of CHS by DNFB resulted in significantly accelerated ear swelling in the mice that had been transferred with T cells from LAIR-1-Ig-treated mice, compared with those from control Ig-treated mice (FIG. 3c). This result also supports inhibitory functions of LAIR-1 on hapten-specific T cells in the sensitization of CHS. Next, to assess LAIR-1 functions in the elicitation phase of CHS, draining LN cells from DNFB-sensitized mice were adoptively transferred into the recipient mice that were pretreated with either LAIR-1-Ig or control protein. After elicitation with DNFB, LAIR-1-Ig-treated recipient mice showed significantly exacerbated ear swelling compared to those treated with control Ig (FIG. 3d).

In addition, LAIR-1 expression was detected on the effector T cells infiltrating in the earlobe after DNFB challenge (FIG. 3e). Altogether, these findings suggest that on hapten-specific effector T cells, LAIR-1 plays n inhibitory role in the elicitation of CHS. LAIR-1 signal inhibits T-cell responses in association with G0/G1 cell cycle arrest.

### Example 4

### Inhibitory Mechanisms of the LAIR-1 Signal in T-Cell Responses

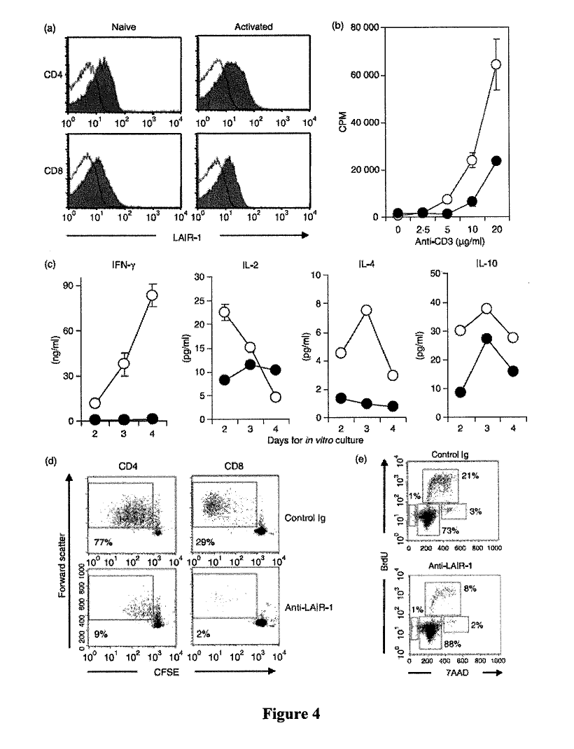

LAIR-1 was weakly, but constitutively, expressed on mouse naïve T cells in both CD4⁺ and CD8⁺ subsets, and its expression level was almost unchanged after stimulation with monoclonal anti-CD3 and monoclonal anti-CD28 (FIG. 4a). Delivery of the LAIR-1 signal by immobilized monoclonal anti-LAIR-1 significantly inhibited T-cell proliferation induced by monoclonal anti-CD3 (FIG. 4b). In addition, the production of IFN-γ, IL-2, IL-4 and IL-10 from the activated T cells was markedly reduced following stimulation with LAIR-1 (FIG. 4c). Both CD4⁺ and CD8⁺ T cells are susceptible to the LAIR-1 inhibitory signal, as cellular division of these subsets was abrogated in the presence of monoclonal anti-LAIR-1 (FIG. 4d). Cell cycle analysis further indicated that the inhibition of T-cell responses by LAIR-1 was associated with cell cycle arrest at the G0/G1 phase, but not with an increased apoptotic population of T cells (FIG. 4e). These results collectively suggest that the LAIR-1 signal inhibits T-cell priming by repressing cell cycle progression, but neither apoptosis induction nor Th1/Th2 deviation.

### Example 5

### Expression and Functions of LAIR-1 in Memory T Cells

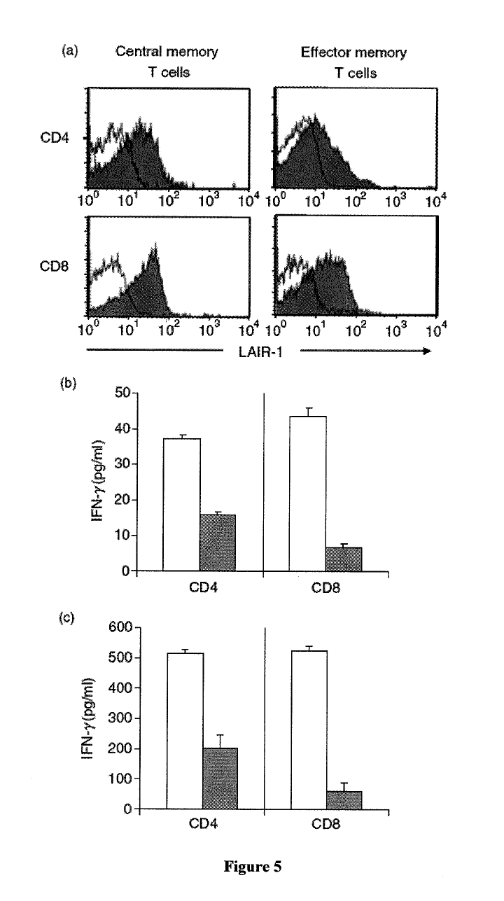

Both central and effector memory T cells express low, but detectable, levels of LAIR-1, while the CD8⁺ T-cell subset expressed LAIR-1 more strongly than CD4⁺ T cells (FIG. 5a). LAIR-1 inhibited monoclonal anti-CD3-induced IFN-γ production in purified CD44^{high} memory T cells as well as in CD44^{low} naïve T cells (FIG. 5b,c). LAIR-1-mediated inhibition of IFN-γ production was observed in both CD4⁺ and CD8⁺ subsets of naïve and memory T cells, while the effects were more striking in CD8⁺ T cells than in CD4⁺ T cells.

### Example 6

### Impaired Cytokine Production of DC in the Presence of LAIR-1 Signal

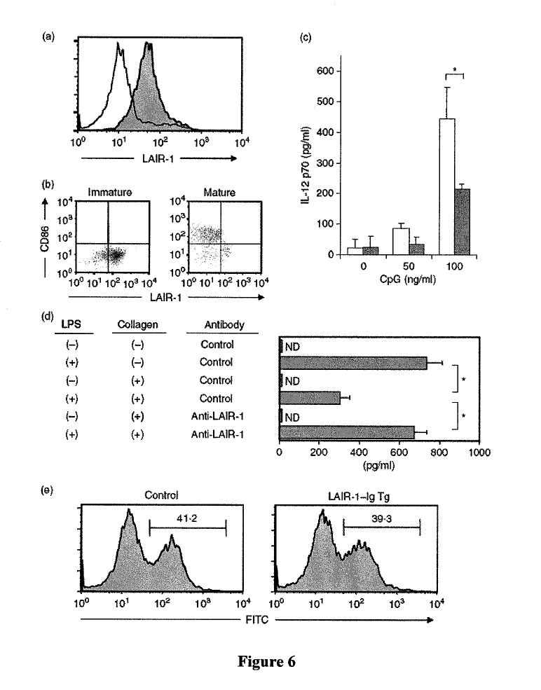

For the sensitization and elicitation of hapten-reactive T cells in CHS, skin antigen-presenting cells (APCs), such as LCs and dermal DCs, play an integral role in antigen acquisition and presentation.32 Whereas LAIR-1 was reported to attenuate GM-CSF receptor signalling and inhibit the differentiation of human monocytes/macrophage into DCs, 9 its direct effects on APC functions remain unknown. To address this, LAIR-1 expression on LCs and DCs was first examined. The majority of freshly harvested LCs and DCs constitutively express LAIR-1 (FIG. 6a,b). LAIR-1 expression on DCs was significantly downregulated along with their activation and maturation by LPS, thus inversely correlating with the upregulation of CD86 (FIG. 6b). The LAIR-1 signal delivered by immobilized monoclonal anti-LAIR-1 significantly inhibited IL-12 p70 production from DCs induced by cytokines and CpG (FIG. 6c). In addition, plate-coated collagen III, one of the functional ligands of LAIR-1,14 also diminished IL-6 production from DCs triggered by LPS stimulation (FIG. 6d). This inhibition was attenuated by blockade of the collagen-LAIR-1 interaction by including soluble monoclonal anti-LAIR-1, supporting the specificity of LAIR-1 inhibitory effects on DCs. By contrast, stimulation of the LAIR-1 signal induced no significant changes in the expression of costimulatory molecules, such as CD80 and CD86, on DCs (data not shown). We also assessed skin DC potential to acquire antigen and migrate to LNs in LAIR-1-Ig transgenic mice. One day after cutaneous application of FITC, the percentage of FITC-positive DC in the draining LNs was comparable between LAIR-1-Ig transgenic mice and control littermates (FIG. 6e). Total DC numbers in the draining LNs of these mice were also comparable (data not shown). Taken together, the LAIR-1 signal regulates DC functions by inhibiting cytokine production, but affecting neither costimulatory molecule expression nor their potential for antigen uptake and migration.

### Example 7

### LAIR-1 Functions on NK Cells In Vitro and In Vivo

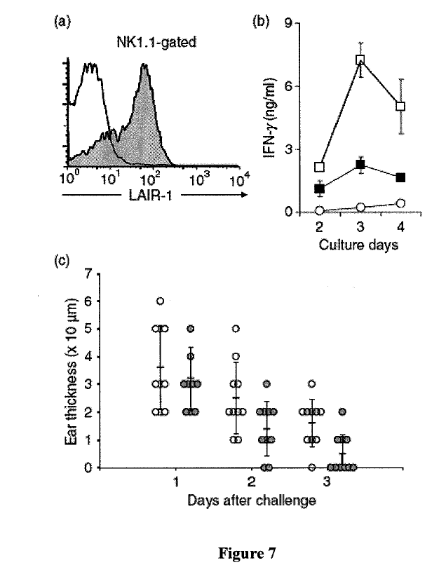

A recent study by O'Leary et al. 20 has revealed that NK cells mediate long-lived, hapten-specific adaptive immunity in CHS independently of T and B lymphocytes. As the inhibitory effects of the LAIR-1 signal in human NK cells have been well demonstrated (1, 2), we attempted to explore whether the effects of LAIR-1 on mouse NK cells regulate the severity of CHS. Initially we examined the expression and functions of LAIR-1 on mouse NK cells in vitro. The majority of NK cells in mouse spleen constitutively express LAIR-1 (FIG. 7a). IFN-γ production from NK cells stimulated with monoclonal anti-NK1.1 and low-dose IL-2 was significantly inhibited in the presence of the LAIR-1 signal (FIG. 7b). In contrast to the in vitro findings, interference of LAIR-1 functions by systemic administration of the LAIR-1-Ig decoy rather decreased the severity of DNFB-induced CHS in B6 RAG2-deficient mice on days 2 and 3 (FIG. 7c).

### Discussion

Whereas an increasing number of studies have demonstrated a potential role for the LAIR-1 inhibitory signal in immune regulation, its pathogenic role in allergic diseases has yet to be explored in vivo. The present study is the first to demonstrate regulatory functions of LAIR-1 in CHS. Interruption of LAIR-1 functions by a LAIR-1-Ig decoy protein led to an enhanced sensitivity to CHS through the acceleration of both sensitization and elicitation phases. The LAIR-1 signal inhibits DC and T-cell responses by repressing the production of cytokines and by inducing G0/G1 cell cycle arrest. The present study indicated three potential mechanisms underlying the inhibitory effects of LAIR-1 in CHS. First, the LAIR-1 signal in DCs inhibits LPS- or CpG-mediated induction of IL-6 and IL-12 (FIG. 6), which are crucial positive regulators in CHS (33, 34). This inhibitory effect seems conceivable as SHP-1, SHP-2 and Csk, which are recruited by the LAIR-1 signal, were reported to negatively regulate Toll-like receptor (TLR)-mediated signals (35-37). As collagens I and III are abundant in the dermis and are capable of delivering the LAIR-1 signal (10, 14) the interaction of these collagens with LAIR-1 at the level of dermal DCs may play an inhibitory role in CHS pathogenesis. Interestingly, the LAIR-1 signal does not equally interfere with multiple arms of DC functions, as the levels of costimulatory expression, antigen-uptake capacity and migration to LNs were maintained at normal levels, even with LAIR-1 stimulation or blockade (FIG. 6 and data not shown). The molecular mechanisms of such selective inhibitory effects remain to be explored. Second, T-cell receptor (TCR)-mediated activation of naïve T cells is significantly repressed by the LAIR-1 signal (FIGS. 4 and 5b). These direct inhibitory effects of LAIR-1 on T cells would be, at least in part, responsible for the regulatory role of LAIR-1 in the sensitization phase of CHS (FIG. 3a-c). A loss of the LAIR-1 signal during the sensitization phase would promote hapten-reactive T-cell priming, in the context of both their number and reactivity, and lead to an increased response to antigen restimulation in vitro or in vivo, as similar phenomena have been demonstrated in other inhibitory co-signals for T cells (38). Third, LAIR-1 shows inhibitory effects in the elicitation phase of CHS (FIG. 3d) and represses memory T-cell responses (FIG. 5). These observations are well concordant with previous studies that the LAIR-1 signal inhibits activation and effector functions of human T cells (2-5

Our current study also provided novel insights into the molecular and cellular mechanisms of LAIR-1-mediated immune inhibitions. First, our results demonstrated that in the presence of TCR stimulation, the LAIR-1 signal enders T cells arrested at the G0/G1 phase, but not apoptotic (FIG. 4e). While a previous study reported that LAIR-1 engagement mediates both programmed cell death and G0/G1 cell cycle arrest by preventing nuclear factor-jB (NF-jB) nuclear translocation (39) this discrepancy probably arises from the difference in the types of cells analyzed (i.e. primary T cells versus myeloid leukaemia cells). As the LAIR-1 signal recruits SHP-1, SHP-2 and Csk, which negatively regulate TCR signal thresholds (40-42), it is conceivable that LAIR-1 represses cell cycle progression, as do other inhibitory co-signalling receptors, including programmed death-1 (PD-1) and cytotoxic T lymphocyte antigen-4 (CTLA-4) (43, 44). Second, our studies revealed that LAIR-1 expression on DCs is downregulated by maturation signals delivered by LPS (FIG. 6b). This result is reminiscent of expression kinetics of other inhibitory receptors on DCs, such as Fcc receptor IIB and dendritic cell immunoreceptor (DCIR) (45, 46) and implies a regulatory role of LAIR-1 in DC homeostasis. Third, LAIR-1 showed negligible effects on Th1/Th2 deviation or suppressor T-cell generation. As shown in FIG. 4c, LAIR-1 signal inhibited IL-4 and IL-10 productions from activated T cells, as well as IFN-γ and IL-2. LAIR-1 signal also decreased the production of IL-10 and transforming growth factor-b (TGF-b) from DCs, and had negligible effects on TGF-b production from activated T cells (data not shown). In addition, the number of CD4⁺ CD25⁺ T regulatory (Treg) cells was comparable between wild-type and LAIR-1-Ig transgenic mice, and LAIR-1-positive lymphocytes showed no suppressor activity on the activation of other T cells (data not shown). These findings suggest that LAIR-1 inhibitory effects on allergic responses are mediated by a direct suppression of DC and T-cell functions, rather than by inducing suppressor T or Treg cells. Our findings provide important insights in the clinical relevance of LAIR-1 functions, which are recently suggested by emerging reports in human diseases. For instance, the serum level of soluble LAIR-1 positively correlates with the severity of haemorrhagic fever with renal syndrome and chronic rejection in kidney transplantation (47). It was also reported that a lack of LAIR-1 expression is associated with high-risk chronic lymphocytic leukaemia patients (48). Finally, an increased level of LAIR-2, a soluble-type LAIR-1 homologue, which could function as a natural competitor for LAIR-1, was detected in the synovial fluid of rheumatoid arthritis patients (49). Exacerbated CHS in our LAIR-1-Ig transgenic model is consistent with these reports and could provide experimental evidence of soluble LAIR-1 or LAIR-2 functions as a decoy immune regulator.

### REFERENCES

1 Meyaard L, Adema G J, Chang C, Woollatt E, Sutherland G R, Lanier L L, Phillips J H. LAIR-1, a novel inhibitory receptorexpressed on human mononuclear leukocytes. Immunity 1997; 7:283-90.
2 Poggi A, Tomasello E, Revello V, Nanni L, Costa P, Moretta L. p40 molecule regulates NK cell activation mediated by NK receptors for HLA class I antigens and TCR-mediated triggering of T lymphocytes. Int Immunol 1997; 9:1271-9.
3 Maasho K, Masilamani M, Valas R, Basu S, Coligan J E, Borrego F. The inhibitory leukocyte-associated Ig-like receptor-1 (LAIR-1) is expressed at high levels by human naive T cells and inhibits TCR mediated activation. Mol Immunol 2005; 42:1521-30.
4 Meyaard L, Hurenkamp J, Clevers H, Lanier L L, Phillips J H. Leukocyte-associated Ig-like receptor-1 functions as an inhibitory receptor on cytotoxic T cells. J Immunol 1999; 162:5800-4. 5 Saverino D, Fabbi M, Merlo A, Ravera G, Grossi C E, Ciccone E. Surface density expression of the leukocyte-associated Ig-like receptor-1 is directly related to inhibition of human T-cell functions. Hum Immunol 2002; 63:534-46.
6 Jansen C A, Cruijsen C W, de Ruiter T, Nanlohy N, Willems N, Janssens-Korpela P L, Meyaard L. Regulated expression of the inhibitory receptor LAIR-1 on human peripheral T cells during T cell activation and differentiation. Eur J Immunol 2007; 37:914-24.
7 van der Vuurst de Vries A R, Clevers H, Logtenberg T, Meyaard L. Leukocyte-associated immunoglobulin-like receptor-1 (LAIR-1) is differentially expressed During human B cell differentiation and inhibits B cell receptor-mediated signaling. Eur J Immunol 1999; 29:3160-7.
8 Verbrugge A, de Ruiter T, Geest C, Coffer P J, Meyaard L. Differential expression of leukocyte-associated Ig-like receptor-1 during neutrophil differentiation and activation. J Leukoc Biol 2006; 79:828-36.
9 Poggi A, Tomasello E, Ferrero E, Zocchi M R, Moretta L. p40/LAIR-1 regulates the differentiation of peripheral blood precursors to dendritic cells induced by granulocyte-monocyte colony-stimulating factor. Eur J Immunol 1998; 28:2086-91.
10 Lebbink R J, de Ruiter T, Kaptijn G J, Bihan D G, Jansen C A, Lenting P J, Meyaard L. Mouse leukocyte-associated Ig-like receptor-1 (mLAIR-1) functions as an inhibitory collagen binding receptor on immune cells. Int Immunol 2007; 19:1011-9.
11 Sathish J G, Johnson K G, Fuller K J, LeRoy F G, Meyaard L, Sims M J, Matthews R J. Constitutive association of SHP-1 with leukocyte-associated Ig-like receptor-1 in human T cells. J Immunol 2001; 166:1763-70.
12 Lebbink R J, de Ruiter T, Verbrugge A, Bril W S, Meyaard L. The mouse homologue of the leukocyte-associated Ig-like receptor-1 is an inhibitory receptor that recruits Src homology region 2-containing protein tyrosine phosphatase (SHP)-2, but not SHP-1. J Immunol 2004; 172:5535-43.
13 Verbrugge A, Rijkers E S, de Ruiter T, Meyaard L. Leukocyte associated Ig-like receptor-1 has SH2 domain-containing phosphatase-independent function and recruits C-terminal Src kinase. Eur J Immunol 2006; 36:190-8.
14 Lebbink R J, de Ruiter T, Adelmeijer J et al. Collagens are functional, high affinity ligands for the inhibitory immune receptor LAIR-1. J Exp Med 2006; 203:1419-25.
15 Skoet R, Zachariae R, Agner T. Contact dermatitis and quality of life: a structured review of the literature. Br J Dermatol 2003; 149:452-6.
16 Grabbe S, Schwarz T. Immunoregulatory mechanisms involved in elicitation of allergic contact hypersensitivity. Immunol Today 1998; 19:37-44.
17 Watanabe H, Unger M, Tuvel B, Wang B, Sauder D N. Contact hypersensitivity: the mechanism of immune responses and T cell balance. J Interferon Cytokine Res 2002; 22:407-12.
18 Saint-Mezard P, Berard F, Dubois B, Kaiserlian D, Nicolas J F. The role of CD4⁺ and CD8⁺ T cells in contact hypersensitivity and allergic contact dermatitis. Eur Dermatol 2004; 14:131-8.
19 Wang B, Feliciani C, Freed I, Cai Q, Sauder D N. Insights into molecular mechanisms of contact hypersensitivity gained from gene knockout studies. J Leukoc Biol 2001; 70:185-91.
20 O'Leary J G, Goodarzi M, Drayton D L, von Andrian U H. T cell and B cell-independent adaptive immunity mediated by natural killer cells. Nat Immunol 2006; 7:507-16.
21 Gelse K, Poschl E, Aigner T. Collagens-structure, function, and biosynthesis. Adv Drug Deliv Rev 2003; 55:1531-46.
22 de Fougerolles A R, Sprague A G, Nickerson-Nutter C L et al. Regulation of inflammation by collagen-binding integrins alpha1-beta1 and alpha2beta1 in models of hypersensitivity and arthritis. J Clin Invest 2000; 105:721-9.
23 Chapoval A I, Zhu G, Chen L. Immunoglobulin fusion proteins as a tool for evaluation of T-cell costimulatory molecules. Mol Biotechnol 2002; 21:259-64.
24 Zhu G, Flies D B, Tamada K, Sun Y, Rodriguez M, Fu Y X, Chen L. Progressive depletion of peripheral B lymphocytes in 4-1BB (CD137) ligand/I-Ealpha)-transgenic mice. J Immunol 2001; 167:2671-6.
25 Choi I H, Zhu G, Sica G L et al. Genomic organization and expression analysis of B7-H4, an immune inhibitory molecule of the B7 family. J Immunol 2003; 171:4650-4.
26 Wilcox R A, Flies D B, Zhu G et al. Provision of antigen and CD 137 signaling breaks immunological ignorance, promoting regression of poorly immunogenic tumors. J Clin Invest 2002; 109:651-9.
27 Wang B, Fujisawa H, Zhuang L et al. CD4⁺ Th1 and CD8⁺ type 1 cytotoxic T cells both play a crucial role in the full development of contact hypersensitivity. Immunol 2000; 165:6783-90.
28 Koch F, Kampgen E, Schuler G, Romani N. Isolation, enrichment, and culture of murine epidermal langerhans cells. In: Robinson S E, Stagg A J, eds. Dendritic Cell Protocols. Totowa, N.J.: Humana Press Inc, 2001:43-62.
29 Wilcox R A, Chapoval A I, Gorski K S et al. Cutting edge: expression of functional CD137 receptor by dendritic cells. J Immunol 2002; 168:4262-7.
30 Hochrein H, O'Keeffe M, Luft T, Vandenabeele S, Grumont R J, Maraskovsky E, Shortman K. Interleukin (IL)-4 is a major regulatory cytokine governing bioactive IL-12 production by mouse and human dendritic cells. J Exp Med 2000; 192:823-33.
31 Wilcox R A, Tamada K, Strome S E, Chen L. Signaling through NK cell-associated CD137 promotes both helper function for CD8⁺ cytolytic T cells and responsiveness to IL-2 but not cytolytic activity. J Immunol 2002; 169:4230-6.
32 Bennett C L, van Rijn E, Jung S, Inaba K, Steinman R M, Kapsenberg M L, Clausen B E. Inducible ablation of mouse Langerhans cells diminishes but fails to abrogate contact hypersensitivity. J Cell Biol 2005; 169:569-76.
33. Hope J C, Campbell F, Hopkins S J. Deficiency of IL-2 or IL-6 reduces lymphocyte proliferation, but only IL-6 deficiency decreases the contact hypersensitivity response. Eur J Immunol 2000; 30:197-203.
34. Riemann H, Schwarz A, Grabbe S et al. Neutralization of IL-12 in vivo prevents induction of contact hypersensitivity and induces hapten-specific tolerance. J Immunol 1996; 156:1799-803.
35 Hardin A O, Meals E A, Yi T, Knapp K M, English B K. SHP-1 inhibits LPS-mediated TNF and iNOS production in murine macrophages. Biochem Biophys Res Commun 2006; 342:547-55.
36 Smith R E, Patel V, Seatter S D et al. A novel MyD-1 (SIRP-1alpha) signaling pathway that inhibits LPS-induced TNFalpha production by monocytes. Blood 2003; 102:2532-40.
37 Iwabuchi K, Hatakeyama S, Takahashi A et al. Csk overexpression reduces several monokines and nitric oxide productions but enhances prostaglandin E2 production in response to lipopolysaccharide in the macrophage cell line J774A.1. Eur J Immunol 1997; 27:742-9.
38 Tsushima F, Yao S, Shin T et al. Interaction between B7-H1 and PD-1 determines initiation and reversal of T-cell energy. Blood 2007; 110:180-5.
39 Poggi A, Pellegatta F, Leone B E, Moretta L, Zocchi M R. Engagement of the leukocyte-associated Ig-like receptor-1 induces programmed cell death and prevents NF-kappaB nuclear translocation in human myeloid leukemias. Eur J Immunol 2000; 30:2751-8.
40 Zhang J, Somani A K, Yuen D, Yang Y, Love P E, Siminovitch K A. Involvement of the SHP-1 tyrosine phosphatase in regulation of T cell selection. J Immunol 1999; 163:3012-21.
41 Newman D K, Hamilton C, Newman P J. Inhibition of antigenreceptor signaling by Platelet Endothelial Cell Adhesion Molecule-1 (CD31) requires functional ITIMs, SHP-2, and p56(lck). Blood 2001; 97:2351-7.
42 Chow L M, Fournel M, Davidson D, Veillette A. Negative regulation of T-cell receptor signalling by tyrosine protein kinase p50csk. Nature 1993; 365:156-60.
43 Latchman Y, Wood C R, Chernova T et al. PD-L2 is a second ligand for PD-1 and inhibits T cell activation. Nat Immunol 2001; 2:261-8.
44 Chuang E, Lee K M, Robbins M D et al. Regulation of cytotoxic T lymphocyte-associated molecule-4 by Src kinases. J Immunol 1999; 162:1270-7.
Boruchov A M, Heller G, Veri M C, Bonvini E, Ravetch J V, Young J W. Activating and inhibitory IgG Fc receptors on human DCs mediate opposing functions. J Clin Invest 2005; 115:2914-23.
46 Bates E E, Fournier N, Garcia E et al. APCs express DCIR, a novel C-type lectin surface receptor containing an immunoreceptor tyrosine-based inhibitory motif. Immunol 1999; 163:1973-83.
47 Ouyang W, Xue J, Liu J et al. Establishment of an ELISA system for determining soluble LAIR-1 levels in sera of patients with HFRS and kidney transplant. J Immunol Methods 2004; 292:109-17.
48 Poggi A, Catellani S, Bruzzone A, Caligaris-Cappio F, Gobbi M, Zocchi M R. Lack of the leukocyte-associated Ig-like receptor-1 expression in high-risk chronic lymphocytic leukaemia results in the absence of a negative signal regulating kinase activation and cell division. Leukemia 2008; 22: 980-8.
49 Lebbink R J, van den Berg M C, de Ruiter T, Raynal N, van Roon, J A, Lenting P J, Jin B, Meyaard L. The Soluble leukocyte-associated Ig-like receptor (LAIR)-2 antagonizes the collagen/LAIR-1 inhibitory immune interaction. J Immunol 2008; 180:1662-9.

1. A monoclonal antibody that specifically interacts with a component of and stimulates a LAIR-1 expression pathway in a LAIR-1 expressing immune cell.

2. The monoclonal antibody of claim 1 that is an anti-LAIR-1 antibody.

3. A pharmaceutical composition comprising the monoclonal antibody of claim 1.

4. The monoclonal antibody of claim 1, wherein the LAIR-1 expression pathway is mammalian.

5. The monoclonal antibody of claim 4, wherein the LAIR-1 expression pathway is human.

6. A pharmaceutical composition comprising monoclonal anti-LAIR-1 antibody.

7. The pharmaceutical composition of claim 6 wherein the concentration of monoclonal anti-LAIR-1 is between 0.1 ng/ml and 100 mg/ml.

8. The pharmaceutical composition of claim 6 that comprises monoclonal antiLAIR-1 antibody diluted in saline at the concentration of 1-25 mg/ml.

9. The pharmaceutical composition of claim 6 that comprises monoclonal antiLAIR-1 antibody formulated as an ointment suitable for topical administration in mineral oil, paraffin, propylene carbonate, white petrolatum and/or white wax for topical administration.

10. The pharmaceutical composition of claim 6 that is formulated for topical administration.

11. A method of treating a contact dermatitis caused by an allergen comprising administering the pharmaceutical composition of claim 3 to a subject in need of treatment.

12. The method of claim 11 wherein the subject is a human.

13. The pharmaceutical composition of claim 6, further comprising at least one diluted, excipient or carrier.

14. A pharmaceutical composition comprising the monoclonal antibody of claim 2.
